(19) United States
(12) Patent Application Publication
(10) Pub. No.: US 2023/0165665 A1
(43) Pub. Date: Jun. 1, 2023

(54) DENTAL ALIGNMENT DEVICE AND METHOD

(71) Applicant: SELF-IE ENTERPRISE PRIVATE LIMITED, Hillview Green (SG)

(72) Inventors: Joey DONOVAN, Ballinhassig (IE); William Won Gon PARK, London (GB)

(21) Appl. No.: 17/995,774

(22) PCT Filed: Apr. 7, 2021
(86) PCT No.: PCT/IB2021/052909

§ 371 Date: Oct. 7, 2022

Related U.S. Application Data
(60) Provisional application No. 63/006,972, filed on Apr. 8, 2020.

(51) Int. Cl. A61C 9/00; A61K 6/90

(52) U.S. Cl. CPC A61C 9/0006; A61K 6/90; A61C 9/0026

(57) ABSTRACT

A dental impression kit and method is provided. The kit includes a single-use container filled with a polyvinyl siloxane (PVS) base, a single-use container filled with a PVS catalyst, a dispenser filled with a light body PVS, an impression tray having a curved channel sized and shaped to surround one of an upper or lower dental arch and an instruction packet. The method includes opening the single-use containers, and mixing the PVS base and PVS catalyst to form a PVS mixture. The method includes pressing the PVS mixture into the impression tray, pressing the impression tray onto one of an upper or lower dental arch to form a first dental impression. The method includes opening the single use dispenser, ejecting the light body PVS over the first dental impression, and reseating the impression tray to form a second dental impression.

### RELATED APPLICATIONS

This application claims priority to U.S. Provisional Application No. 63,006,972, filed on Apr. 8, 2020, the entire disclosure of which is incorporated herein by reference.

### BACKGROUND

For many years, orthodontists have used traditional metal braces to treat malocclusion in patients. For traditional metal braces, metal brackets are adhered to each tooth, and a metal wire is threaded through the brackets. An orthodontist can adjust the metal wire in iterative steps to bring teeth into an ideal alignment. While conventional metal braces are cost-effective, they are highly visible in the mouth of the patient. In response to the aesthetic concerns associated with metal braces, clear alignment trays that sequentially align teeth were developed. For example, the patient can receive a series of alignment trays, which fit over the entire maxillary arch or the entire mandibular arch, and each iterative tray is structured to shift the teeth slightly more into an ideal alignment.

To produce the alignment trays, a dental impression must be taken of one or both of the maxillary arch and the mandibular arch. First, a dental care provider mixes together or provides an impression material that is moldable around the hard and soft tissue features of the patient's mouth. The dental care provider then fills a dental impression tray with the impression material. The impression tray is pressed over the upper or lower teeth until the impression material sufficiently hardens, usually after a minute or two, to create a negative imprint of the features of the mouth. The impression tray is then removed from the patient's mouth. The dental impression can be used to produce a positive rendering of the patient's teeth, and the surrounding soft tissue, for alignment tray customization. Three-dimensional computer modeling can be used to design each tray to slowly align the patient's teeth overtime.

Although alignment trays are very effective when indicated, it can be inconvenient for patients to travel to a dental care provider for the impression taking process. To solve this problem, alignment tray developers now sell direct to consumers by providing a kit for the recipient to make their own dental impression at home using a heavy body impression material. However, because the impression process requires precise timing and because the impression must reflect very accurate details to be effective, the at-home impression process with a heavy body impression material can have a high rate of failure.

### SUMMARY

Some embodiments provide a kit for forming a dental impression. The kit can include a first single-use container filled with a polyvinyl siloxane (PVS) base, a first single-use container filled with a PVS catalyst, a single-use dispenser filled with a low-viscosity PVS, an impression tray having at least one curved channel sized and shaped to surround one of an upper or lower dental arch, and an instruction packet. The instruction packet can include instructions to set a timer, mix the PVS base and the PVS catalyst to form a PVS mixture, press the PVS mixture into the impression tray, and press the impression tray filled with the PVS mixture onto one of the upper or the lower dental arch to form a first dental impression. The instruction packet can also include instructions to eject the low-viscosity PVS over the first dental impression, and reseat the impression tray loaded with the low-viscosity PVS onto one of the upper or the lower dental arch to form a second dental impression.

In some forms, the impression tray has two curved channels, one curved channel being sized and shaped to surround the upper dental arch and the other curved channel being sized and shaped to surround the lower dental arch. The kit can include at least two first single-use containers filled with the PVS base and at least two second single-use containers filled with the PVS catalyst. The first single-use container filled with the PVS base can be a first barrel of a double barrel syringe and the first single-use container filled with the PVS catalyst can be a second barrel of the double barrel syringe. The kit can further include at least one p air of fluid resistant gloves and/or a cheek retractor.

Some embodiments provide a method of forming a dental impression. The method can include providing a kit having a first single-use container filled with a PVS base, a second single-use container filled with a PVS catalyst, an impression tray, and a single-use dispenser containing a low-viscosity PVS. The method can include opening the first single-use container, opening the second single-use container, and mixing the PVS base and PVS catalyst to form a PVS mixture. The method can include pressing the PVS mixture into the impression tray and pressing the impression tray filled with the PVS mixture onto one of an upper or lower dental arch to form a first dental impression. The method can include opening the single-use dispenser, ejecting the low-viscosity PVS over the first dental impression, and reseating the impression tray onto one of the upper dental arch or the lower dental arch to form a second dental impression. In some forms, the method can include removing the impression tray from the one of the upper dental arch or the lower dental arch about 1 minute to about 4 minutes after reseating the impression tray to form the second dental impression.

In some forms, the method includes dividing the PVS mixture into a first mixture and a second mixture, rolling the first mixture into a first elongated, tubular shape and rolling the second mixture into a second elongated, tubular shape, and measuring a length of the first elongated, tubular shape and the second elongated, tubular shape. The method can also include dividing the PVS mixture into a first mixture and a second mixture within 20 seconds of manually mixing the PVS base and PVS catalyst.

In some forms, the impression tray includes a top side and a bottom side, and the method includes pressing the first mixture into the top side of the impression tray and pre s sing the second mixture into the bottom side of the impression tray. The method can include setting a timer before mixing the PVS base with the PVS catalyst. The method can include holding the impression tray in place for at least three minutes before removing the impression tray. The method can include ejecting the low-viscosity PVS over the first dental impression within 45 seconds of opening the single-use dispenser. The method can include placing a cheek retractor into a mouth of a user. The method can include taking a picture of the mouth of the user after the cheek retractor is placed in the mouth of the user.

### DESCRIPTION OF THE DRAWINGS

FIG. 1 is a schematic view of a kit for forming a dental impression according to an embodiment;

FIG. 2A is a front elevational view of a first single-use container of the kit of FIG. 1;

FIG. 2B is a front elevational view of a second single-use container of the kit of FIG. 1;

FIG. 3 is a side elevational view of a single-use dispenser of the kit of FIG. 1;

FIG. 4A is a top plan view of an impression tray of the kit of FIG. 1;

FIG. 4B is a rear elevational view of the impression tray of FIG. 4A;

FIG. 5A is a top plan view of an alternative impression tray of the kit of FIG. 1;

FIG. 5B is an isometric view of the alternative impression tray of FIG. 5A;

FIG. 6A is a top isometric view of an alternative impression tray of the kit of FIG. 1;

FIG. 6B is an exploded view of the alternative impression tray of FIG. 6A;

FIG. 7 is an isometric view of an alternative impression tray of the kit of FIG. 1;

FIG. 8 is a schematic view of an instruction packet of the kit of FIG. 1; and

FIG. 9 is a block diagram of a method of forming a dental impression according to an embodiment.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will b e readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As mentioned above, conventional at-home dental impression kits can lead recipients to produce impressions that are unsatisfactory for fabricating dental alignment trays. Some at-home dental impression kits provide an impression method that uses a single, high viscosity impression material such as heavy body polyvinyl siloxane (PVS) (e.g., Aquasil™ Soft Putty), addition-curing PVS putty (e.g., DMG™ Honigum Pro™ Heavy), or condensation curing PVS (e.g. Penton™ Siloflex™ Plus Putty). However, used alone, due to the high viscosity of these materials, they are unable to flow sufficiently around teeth to capture details of the hard and soft tissues. The resulting impression can have insufficient resolution to produce an acceptable alignment tray.

A two-step putty wash technique was developed to create higher resolution dental impression models. In this method, a dental care provider uses a lower viscosity impression material, such as light body PVS, e.g. Aquasil™ Ultra Low Viscosity Fast Set or 3M™ Imprint™ 3 Quick Step Light Body VPS Impression Material, in addition to a high viscosity impression material.

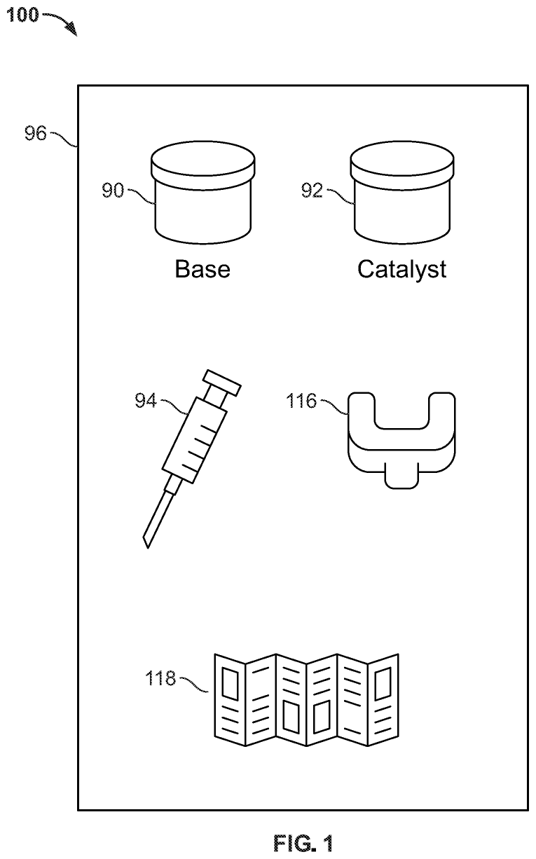

FIGS. 1-5 illustrate a kit 100 for forming dental impression according to an embodiment of the disclosure. The kit 100 includes a variety of parts to assist in, and provide for, the preparation of an accurate dental impression(s). For example, the kit 100 includes a single-use container 90 filled with a polyvinyl siloxane (PVS) base 110 and a single-use container 92 filled with a PVS catalyst 112. The kit 100 also includes a single-use dispenser 94 filled with a light body PVS 114, and an impression tray 116. An instruction packet 118 having easy to read text and descriptive graphics is also provided with the kit 100 to guide users in the dental impression process. The individual parts of the kit 100 are described further below and are provided in a single packaging 96. Although not shown, the kit 100 can include one or more additional parts, such as a cheek retractor, fluid resistant gloves, a timer, a ruler, a towel or washcloth, a toothbrush, dental floss, and a mirror, among other assistive dental tools, such as dental tools which are conventionally known.

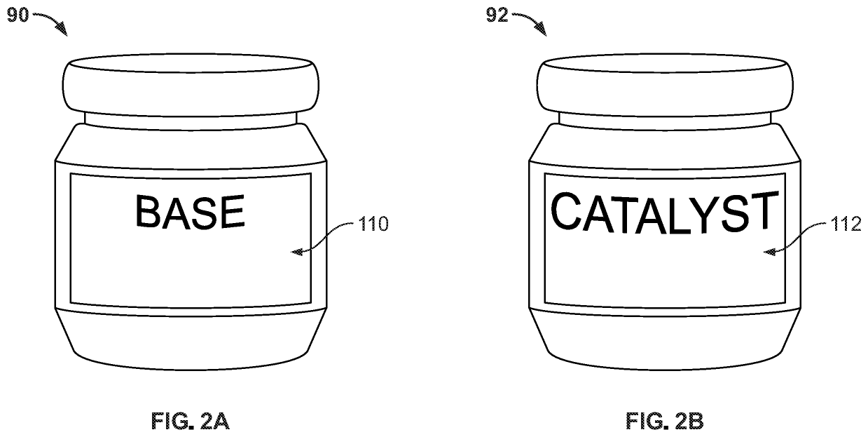

FIGS. 2A and 2B illustrate the single use-containers 90, 92 that are filled with the PVS base 110 and the PVS catalyst 112, respectively. The PVS base 110 and the PVS catalyst 112 can be colored differently from each other, helping users distinguish between them. Additionally, the container 90 and the container 92 can be colored to correspond with the color of respective substance contained therein, or colored in a manner that generally distinguishes between the containers 90, 92. Each container 90, 92 can be provided in the form of a jar with a screw off or pop-off lid that is sealed from the environment outside of the jar. The containers 90, 92 can also be provided in the form of any other air tight container, particularly as a disposable, single-use container such as a sealed plastic pouch. In some forms, the container 90 comprises a first barrel of a double-barreled syringe and the container 92 comprises a second barrel of the double-barreled syringe (not shown).

In some forms, the kit 100 includes multiple containers 90, 92. For example, one set of containers 90, 92 can provide a first impression forming attempt for both the upper and lower arches. However, the kit 100 can also include an additional set of containers 90, 92 that may b e used for another impression forming attempt for both the upper and lower arches in the event that the first attempt is unsuccessful. In some forms, the kit 100 can include multiple sets of single use containers 90, 92 for a third, fourth, or more attempts at impression forming.

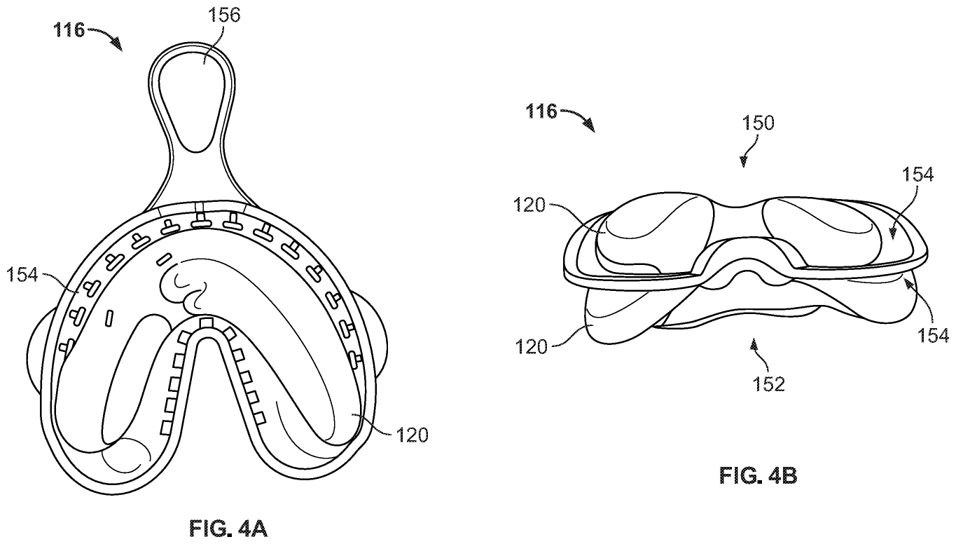

The amount of the PVS base 110 and the PVS catalyst 112 provided within the containers 90 and 92, respectively, is provided in the required ratio so that a mixture containing heavy body PVS 120 (shown in FIGS. 4A, 4B) is formed when substantially the entire contents of the single use containers 90, 92 are mixed together. For example, the single use container 90 can be filled with about a 25 gram mass of the PVS base 110, and the single use container 92 can be filled with about a 25 gram mass of the PVS catalyst 112. When substantially all of the contents of each container 90, 92 are thoroughly mixed together, a 50 gram mass of the mixture containing heavy body PVS mixture 120 is formed, having the material properties suitable for forming a dental impression.

The PVS mixture 120 is elastically deformable and moldable around the teeth and soft tissues of the mouth for a short period of time, e.g. a few minutes, while also providing the dimensional stability to retain a negative imprint of at least a portion of the anatomical features of the mouth. For example, the resulting PVS mixture 120 can be classified as a Type 0 or Type 1 elastomeric impression material (ISO4823.2015) with a linear dimensional change of less than 0.1%, have a maximum strain in compression of 2.5% to 4.5%, and have a greater than 99% recovery from deformation. The PVS mixture 120 can have a working time value of about 1 minute to about 3 minutes, an intra-oral set time value of about 1 minute to about 3 minutes, a specific gravity value of about 1.0 gr/mL to about 2.0 gr/mL, and a hardness value of about 50 to about 60 Shore A. In some forms, the working time of the PVS mixture 120 is controlled in part by thermal activation of the putty setting chemistry, which occurs at body temperature and allows a longer working time. Accordingly, if the user delays placing the PVS mixture 120 in their mouth, it will not subtract from the working time. The PVS mixture can also have a color of blue or dark blue and a life of about 2 years from release to expiration.

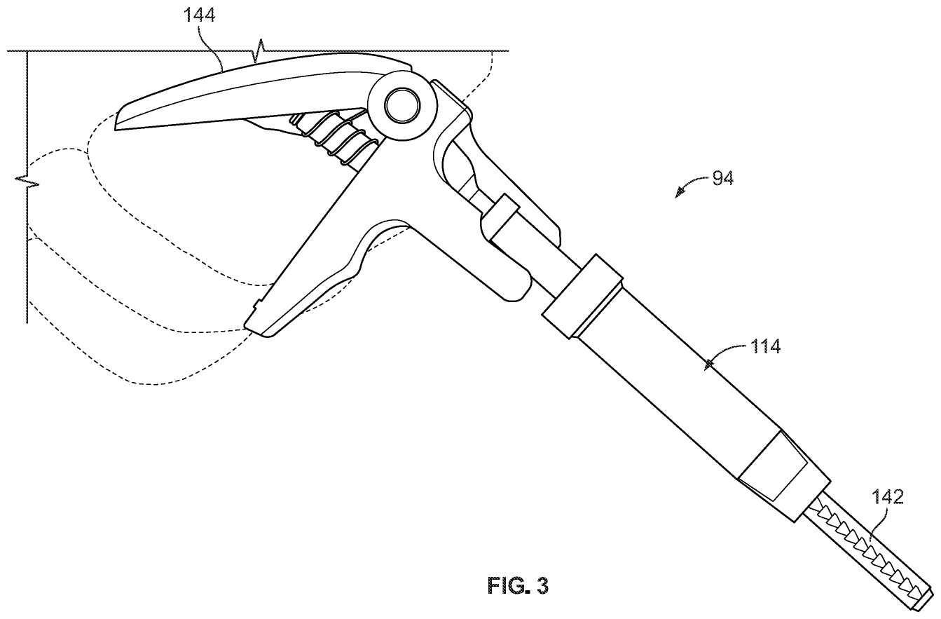

FIG. 3 illustrates the dispenser 94 filled with the light body PVS 114 according to an embodiment of the disclosure. The dispenser 94 can be configured as a syringe having a tube, a nozzle, a twist-off cap, and a piston, which can be actuated to eject the contents of the tube. The twist-off cap can be configured to generate a sound, such as a clicking noise, when it is twisted off to indicate to the user that the dispenser 94 has been successfully opened. The dispenser 94 can also be configured as another air tight container, particularly as a disposable sealed plastic pouch, or a container that allows ejection control of the light body PVS 114. The dispenser 94 contains a quantity of the light body PVS 114 to substantially cover the entire impression tray 116 about 2 times to about 4 times in a thick layer of the light body PVS 114. For example, the dispenser 94 can be filled with about a 5 mL volume of the light body PVS 114.

The dispenser 94 includes a blunt applicator tip 142 for controlling the flow of the light body PVS 114. The dispenser 94 can be configured for a single-use, limited-use, or non-reuse. For example, the dispenser 94 can be removed from the kit 100, opened to allow flow of the light body PVS 114, actuated to eject the light body PVS 114 into the impression tray 116 pursuant to the directions in the instruction packet 118, and then disposed. In some forms, once the dispenser 94 is opened, it cannot be closed for re-use, e.g. one dispenser 94 is only usable with one kit 100. Alternatively, the dispenser 94 can be configured to be closed after use, so as to provide a limited number of uses after opening. Alternatively, the kit 100 can include up to four separate dispensers 94, so as to correspond to four separate dental impression molds—two impressions produced for the upper dental arch and two impressions taken for the lower dental arch.

In some embodiments, an extruder gun 144 can also be provided in the kit 100. The extruder gun 144 has a holster and a spring-assisted trigger, and is configured to receive the dispenser 94. As shown in FIG. 3, the dispenser 94 can be coupled to the extruder gun 144, which can provide spring-assisted ejection of the light body PVS 114.

FIGS. 4A and 4B illustrate the impression tray 116 according to an embodiment of the disclosure. The impression tray 116 is sized and shaped to surround both the upper and the lower dental arches simultaneously. For example, the impression tray 116 includes a top side 150 and a bottom side 152, and can have two curved channels 154. One of the curved channels 154 is sized and shaped corresponding to the upper dental arch and is positioned on the top side 150. The other channel 154 is sized and shaped corresponding to the lower dental arch and is positioned on the bottom side 152. In some forms, the impression tray 116 is adjustable in size to fit a wide range of user mouth dimensions. One advantage of providing the impression tray 116, which corresponds to both the upper and lower arches, is that occlusal or “bite” information about how the teeth meet together can be evaluated. Accordingly, tooth movements can be designed in reference to the additional dimension of how the upper and lower arches relate to one another. Conventional at-home dental impression kits do not provide two-sided impression trays, and, thus, do not allow occlusal information to be evaluated. The impression tray 116 also includes a rigid tab 156 to assist the user in removing the impression tray 116 during the impression forming process, which will be described in depth below.

The impression tray 116 comprises a rigid material such that the impression tray 116 maintains structural integrity when the impression tray 116 is subjected to being pressed against the dental arches of the user or is compressed by the bite of the user during the impression forming process. For example, in some embodiments, the impression tray 116 can comprise polyvinyl chloride (PVC) or high density polyethylene (HDPE). In some forms, the kit 100 can provide two separate impression trays, e.g. one impression tray 116 for the upper dental arch and one impression tray 116 for the lower dental arch. Accordingly, the first of the two impression trays 116 can be sized and shaped to surround the upper dental arch and the second impression tray 116 can be sized and shaped to surround the lower dental arch.

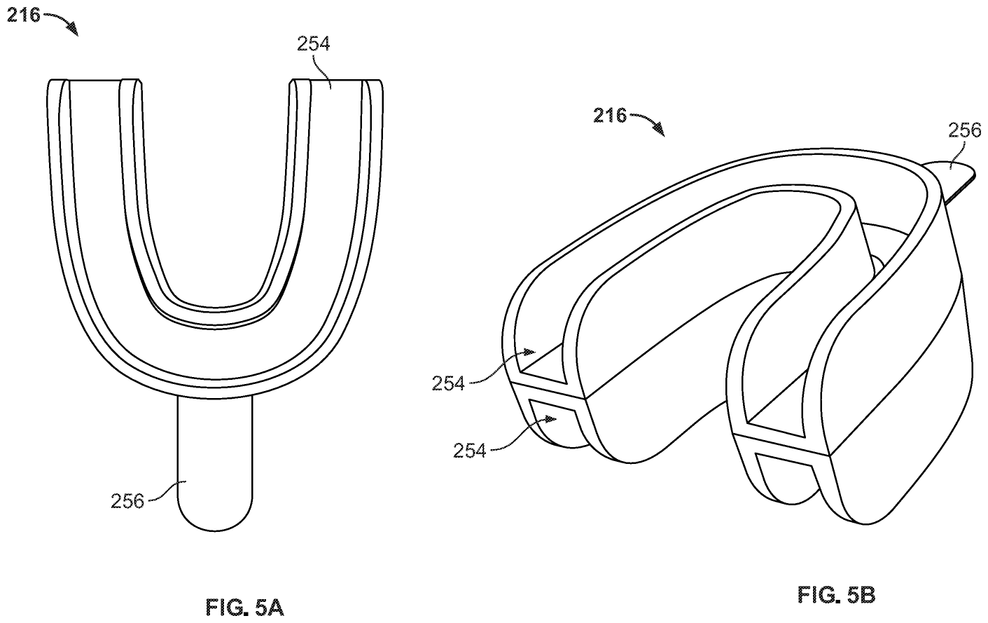

FIGS. 5A and 5B illustrate an alternative impression tray 216. The principles described above with respect to the impression tray 116 are incorporated herein to the description of the impression tray 216. For example, the impression tray 216 includes two curved channels 254 and a rigid tab 256. Also, the impression tray 216 can be comprised of a moldable, deformable, thermoplastic material such as ethylene-vinyl acetate (EVA). Thus, when exposed to heat, the impression tray 216 can be structurally adjusted to assist in the impression forming process.

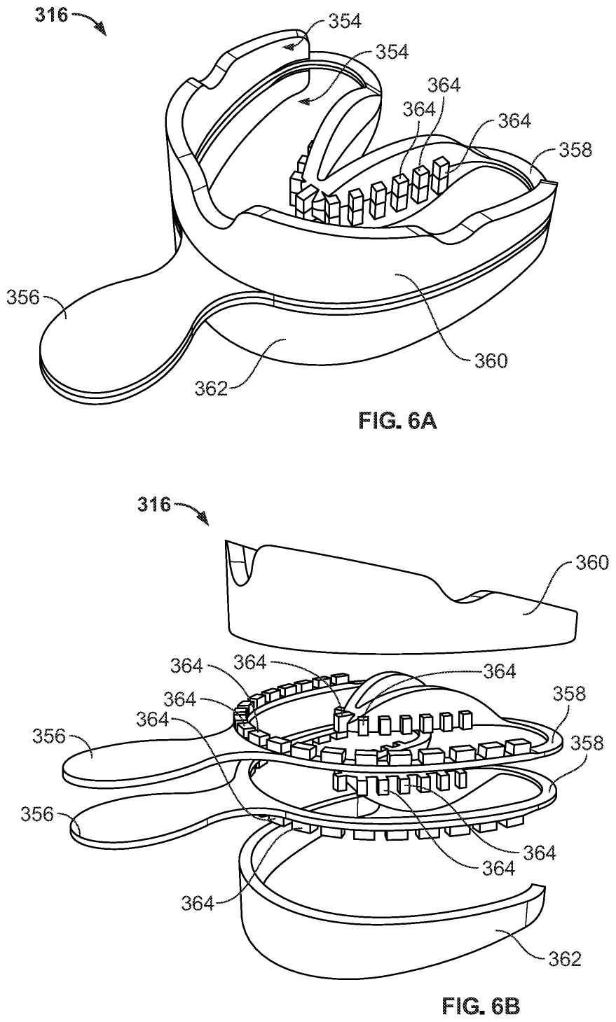

FIGS. 6A and 6B illustrate an alternative impression tray 316. The principles described above with respect to the impression trays 116 and 216 are incorporated herein to the description of the impression tray 316. For example, the impression tray 316 includes two curved channels 354. A rigid tab 356 is provided, however, the rigid tab 356 also forms a frame 358 for the impression tray 316. The rigid tab 356 can be manufactured in two separate pieces and comprise a rigid material such as polystyrene (PS), polyvinyl chloride (PVC), or high density polyethylene (HDPE). In some forms, the rigid tab 356 includes an embossed label such as “up” or “down” to assist the user in identifying the correct orientation of the impression tray 316. The two pieces of the rigid tab 356 are joined together over a mesh layer (not shown), comprising polyamide or similar materials, to separate the two curved channels 354 into corresponding upper and lower dental arch portions. An upper rim 360 and a lower rim 362 are formed onto the frame 358 and comprise moldable, deformable, thermoplastic material such as ethylene-vinyl acetate (EVA). Thus, when exposed to heat, the impression tray 316 can be structurally adjusted to assist in the impression forming process. The frame 358 can include protrusions 364, which can help retain the upper and lower rims 360, 362, as well as the PVS mixture 120.

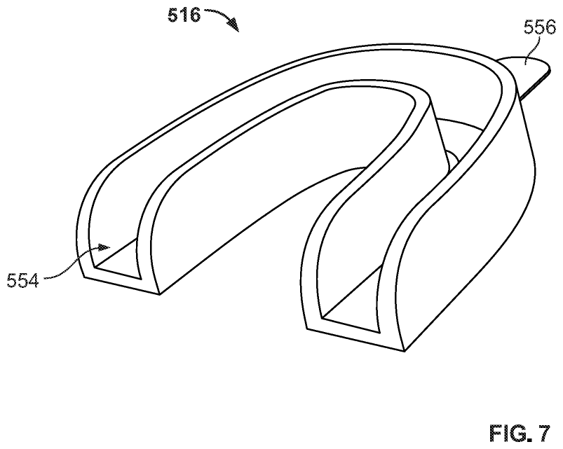

As mentioned above, some embodiments of the kit 100 can include one or more one-sided impression trays such as an impression tray 516 shown in FIG. 7. The principles described for the two-sided impression trays 116, 216, 316 similarly apply to the embodiment having at least one one-sided impression tray 516. For example, the impression tray 516 includes a curved channel 554 and a rigid tab 556. In some forms, the kit 100 can include two one-sided impression trays 516, one for each of the dental arches. In some forms, when two one-sided impression trays 516 are included in the kit 100, each is similarly shaped but sized to a different scale; thus, one of the one-sided impression trays 516 is smaller than the other. In this way, one of the one-sided impression trays 516 can be used with the lower dental arch and one of the one-sided impression trays 516 can be used with the upper dental arch.

The one-sided impression tray 516 can be pressed onto one of the upper or the lower dental arches (rather than the user biting down) to form the dental impressions. The instruction packet 118 can provide information about the magnitude of force that the user should use to press the one-sided impression tray 516 onto the dental arches for steps 420 and 426, which can be selected from a range of about ten percent of the user's maximum strength to the maximum strength with which the user can press on the one-sided impression tray 516. For example, the instruction packet 118 can indicate that the user should apply about fifty percent of their full strength to the one-sided impression tray 516. Alternatively, the instruction packet 118 can indicate that the user should apply their maximum strength, pressing down as hard as they can until the PVS mixture 114 extends over the user's gums.

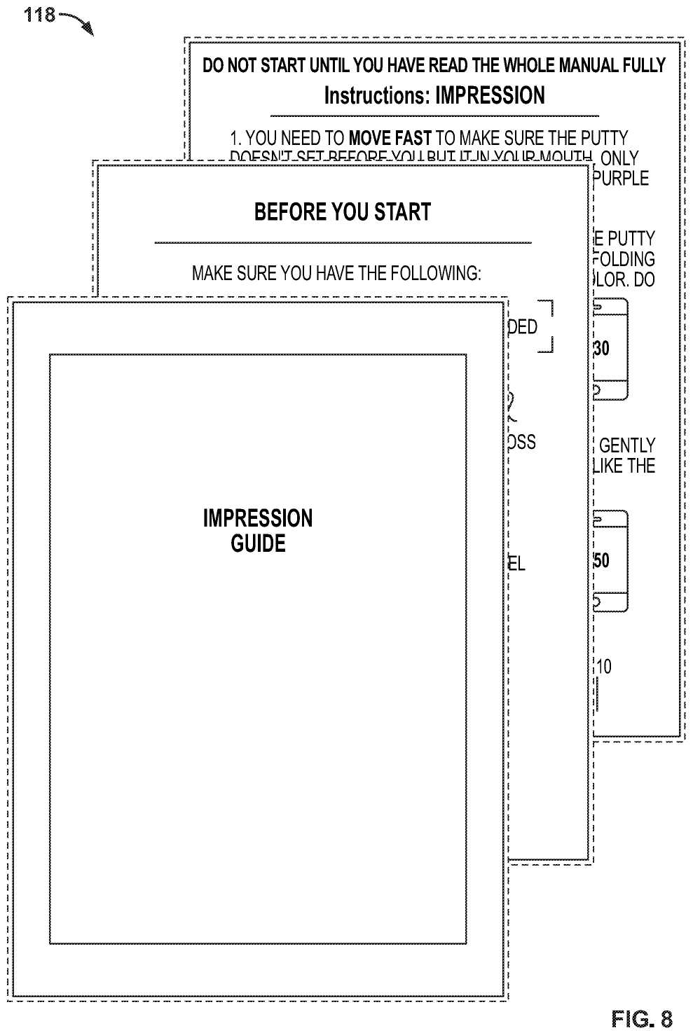

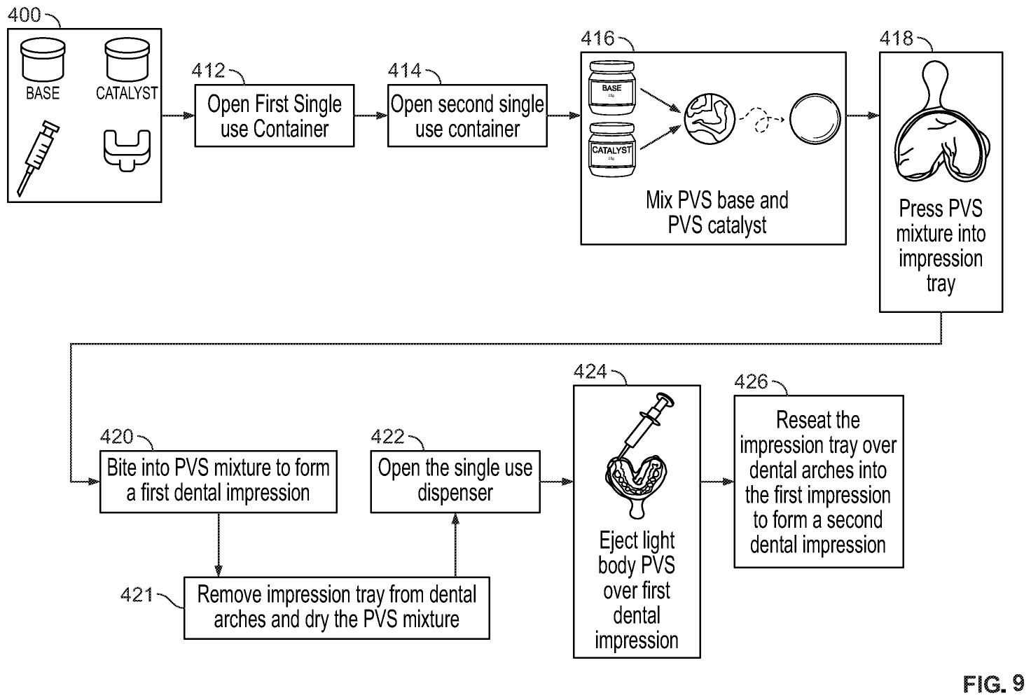

FIG. 8. illustrates an instruction packet 118 according to an embodiment of the disclosure. The instruction packet 118 can include one or more pages that specifically detail the process of forming a dental impression using the kit 100. The instruction packet 118 can include text, diagrams, pictures, figures, machine readable coding, and any other features necessary to aide the user in performing the dental impression process. The instruction packet 118 conveys a method of forming dental impressions, and will include the steps described in detail with respect to FIG. 9 below. In some forms, the instruction packet 118 may be provided in other forms, such as, for example, a digital format (e.g., application provided on a smart phone or other similar device, an instructional video, and the like).

FIG. 9 illustrates a block diagram of a method of forming a dental impression according to an embodiment of the disclosure. In step 400, the kit 100, according to the principles described above, is provided. It should be noted that prior to providing the kit 100, the user's mouth may be measured to assess a number of dimensional aspects of the upper and lower dental arches, such as a mouth width dimension, a mouth height dimension, or a mouth depth dimension so as to provide an appropriately sized impression tray 116 in the provided kit 100. The containers 90 and 92 are opened (steps 412, 414) to access, respectively, the PVS base 110 and the PVS catalyst 112. In step 416, the PVS base 110 and the PVS catalyst 112 are mixed together through manual mixing or by other conventionally known mixing methods. In some forms, a timer can be set prior to step 416, so that a specific timeline can be followed once mixing has started.

For example, because the PVS mixture 120 begins to harden within about one minute, the user can ensure that the steps subsequent the step 416 have commenced within one minute of beginning to mix the PVS base 110 with the PVS catalyst 112. For example, the user can commence step 418 within ten to sixty seconds after beginning to mix the PVS base 110 with the PVS catalyst 112 in step 416. In some forms, the user can commence step 418 within 5, 10, 15, 20, 25, 30, 35, 40, 45, 50, 55, or 60 seconds after beginning to mix the PVS base 110 with the PVS catalyst 112 in step 416. In some embodiments, if the PVS base 110 and the PVS catalyst 112 are provided in different colors, the user can mix the putty together until the PVS mixture 120 presents as a substantially uniform color without streaks. In some forms, the PVS mixture 120 is divided into two parts, and each part is rolled into an elongated tubular shape. Each part of the PVS mixture 120 can be measured against a ruler that can be provided in the kit 100, or the parts can be measured using other conventional measuring methods. The timer can be monitored by the user so that enough time remains after mixing the PVS base 110 with the PVS catalyst 112 to divide and measure the PVS mixture 120 in under one minute.

In step 418, the PVS mixture 120 is pressed into the channels 154 of the impression tray 116. One part of the two parts of the divided PVS mixture 120 can be pressed into the channel 154 on the top side 150 and the other part can be pressed into the channel 154 on the bottom side 152. Thus, the quantity of PVS mixture 120 can provide for the simultaneous formation of both an upper dental arch impression and a lower dental arch impression. In step 420, the user bites down into the PVS mixture 120 of both the top side 150 and the bottom side 152 of the impression tray 116. Meanwhile, the PVS mixture 120 continually hardens, becoming more dimensionally stable to form a first, low resolution dental impression of the upper and lower dental arches.

Once sufficient hardening of the PVS mixture 120 has occurred, the user's dental arches can be carefully removed from the PVS mixture 120 in step 421 by gently agitating or wiggling the impression tray 116. The instruction packet 118 can include an advisory note that the impression tray 116 may be tight or difficult to remove after the PVS mixture 120 has hardened and so the impression tray 116 may require agitation for removal. During the hardening time, the user can monitor the timer to ensure that about two minutes and thirty seconds to about three minutes and thirty seconds have passed before removing the dental arches from the PVS mixture 120. In some forms, the instruction packet 118 can provide a recommended time frame corresponding with step 420, including a recommended time between impressing the dental arches into the PVS mixture 120 to removal of the impression tray 116 and PVS mixture 120 of about 1 minute to about 4 minutes. Once the impression tray 116 is removed, the instruction packet 118 can instruct the user to gently dry the PVS mixture 120, such as with a paper towel or other absorbent material, before proceeding to step 422.

In step 422, the single-use dispenser 94 is opened. The light body PVS 114, which is more flowable than the PVS base 110, the PVS catalyst 112, and the PVS mixture 120, is ejected into and over the first, low resolution dental impression in step 424. The instruction packet 118 can recommend that the user eject the entire contents of the single-use dispenser 94 over the first dental impression and to ensure that the light body PVS 114 is ejected into each tooth of the first, low resolution dental impression. In some forms, the timer can be set to ensure that the light body PVS 114 is ejected within a specified period of time of opening the dispenser 94, such as within one minute. In step 426, the impression tray 116 is reseated over the upper and lower dental arches into the first, low resolution dental impression. Meanwhile, the light body PVS 114 continually hardens, becoming more dimensionally stable to form a second, high resolution dental impression of the upper and lower dental arches in an additive fashion over the first, low resolution dental impression.

Once sufficient hardening of the light body PVS 114 has occurred, the user's dental arches can be carefully removed from the impression tray 116 to preserve the fine details of the user's anatomical dental features. For example, the user can monitor the timer to ensure that about two minutes and thirty seconds to about three minutes and thirty seconds have passed before removing the dental arches from the impression tray 116. In some forms, the instruction packet 118 can provide a recommended time frame corresponding with step 426, including a recommended time between impressing the dental arches into the light body PVS 114 to removal of the impression tray 116 of about 1 minute to about 4 minutes. After the dental impressions have been formed, the user can send the impressions to a manufacturer for the production of sequential alignment trays. The user can also provide the manufacturer with dental photos taken with a personal camera. The cheek retractor mentioned above can be included in the kit 100 to provide a clear view of the anatomical features of the mouth. While the method depicted in FIG. 8 has been described with respect to impression tray 116, the method is not to be limited to use with the impression tray 116, but can also apply to like parts with respect to impression trays 216, 316, and 516.

In some embodiments that use the alternative impression trays 216 or 316, a modified kit and method of forming a dental impression is provided. The modified kit can include the alternative impression tray 216 or 316 and one or both of the light body PVS 114 or the PVS base 110 and the PVS catalyst 112. In some forms, the alternative impression tray 216, 316 itself serves as the first, low resolution dental impression. For example, the method of forming a dental impression may include the additional step of heating the alternative impression tray 216, 316 and molding the alternative impression tray 216, 316 over one or both of the upper or lower dental arches to form a first, low resolution dental impression with dimensional stability. Next, the light body PVS 114 can be ejected into the alternative impression tray 216, 316 to form the second, high resolution dental impression. Alternatively, the PVS mixture 120 (formed by mixing the PVS base 110 and the PVS catalyst 112 as described above) can be placed into the alternative impression tray 216, 316 to form the second, high resolution dental impression. Accordingly, the alternative impression tray 216,316 itself forms the first, low resolution dental impression and one of the light body PVS 114 or the PVS mixture 120 is used to form the second, higher resolution dental impression.

For embodiments of the kit 100 including multiple sets of containers 90, 92 and dispensers 94, the method described above can be repeated to form one or more additional sets of dental impressions for each of the upper and lower dental arches. In some embodiments, a heavy body impression putty, which does not require the mixing of a catalyst and a base, can be used in place of the PVS mixture 120.

Thus, an improved dental impression kit and method is provided by this disclosure. Providing a light body PVS, individual containers and dispensers for easy use, certain impression tray features, and easy to follow instructions that detail impression methods and timing en sure s better resolution, accuracy, and precision in dental impression formation. Test data has shown that the kit 100 improves the usability of dental impressions taken directly by the consumer. Thirty upper and thirty lower impressions were taken by test consumers using a conventional kit and method, and thirty upper and thirty lower impressions were taken by test consumers using the kit 100 and method provided by this disclosure. Only 21% of the impressions produced using the conventional kit and method had sufficient accuracy for developing sequential alignment trays for tooth alignment. Alternatively, 54% of the impressions produced using the kit 100 and method provided herein had sufficient accuracy for developing sequential alignment trays for tooth alignment. Accordingly, the kit 100 and method of this disclosure provided a 157% increase in at-home dental impression usability compared to conventional methods.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A kit for forming a dental impression, the kit comprising:
a first single-use container filled with a polyvinyl siloxane (PVS) base;
a second single-use container filled with a PVS catalyst;
a single-use dispenser filled with a light body PVS;
an impression tray having at least one curved channel sized and shaped to surround one of an upper or lower dental arch; and
an instruction packet, including instructions to:
set a timer,
mix the PVS base and the PVS catalyst to form a PVS mixture,
press the PVS mixture into the impression tray,
press the impression tray filled with the PVS mixture onto one of the upper dental arch or the lower dental arch to form a first dental impression,
eject the light body PVS over the first dental impression, and
reseat the impression tray loaded with the light body PVS onto one of the upper or the lower dental arch to form a second dental impression.

2. The kit of claim 1, further comprising at least two first single-use containers filled with the PVS base and at least two second single-use containers filled with the PVS catalyst.

3. The kit of claim 1, wherein the first single-use container filled with the PVS base is a first barrel of a double barrel syringe and the second single-use container filled with the PVS catalyst is a second barrel of the double barrel syringe.

4. (canceled)

5. (canceled)

6. The kit of claim 1, wherein a viscosity of the PVS mixture is greater than a viscosity of the light body PVS.

7. The kit of claim 1, wherein the instruction packet provides a recommended time value to elapse after pressing the impression tray filled with the PVS mixture onto one of the upper dental arch or the lower dental arch.

8. The kit of claim 7, wherein the instruction packet further includes instructions to agitate the impression tray during removal after the recommended time value has elapsed.

9. The kit of claim 7, wherein the recommended time value is between about 1 minute and about 4 minutes.

10. A method of forming a dental impression, the method comprising:
providing a kit having a first single-use container filled with a polyvinyl siloxane (PVS) base, a second single-use container filled with a PVS catalyst, an impression tray, and a single-use dispenser containing a light body PVS;
opening the first single-use container;
opening the second single-use container;
mixing the PVS base and PVS catalyst to form a PVS mixture;
pressing the PVS mixture into the impression tray;
pressing the impression tray filled with the PVS mixture onto one of an upper dental arch or a lower dental arch to form a first dental impression;
opening the single-use dispenser;
ejecting the light body PVS over the first dental impression; and
reseating the impression tray onto one of the upper dental arch or the lower dental arch to form a second dental impression.

11. The method of claim 10, further comprising the step of setting a timer before mixing the PVS base with the PVS catalyst.

12. The method of claim 10, wherein the PVS base is provided in a first color different from a second color of the PVS catalyst.

13. The method of claim 12, wherein the PVS base and the PVS catalyst are mixed together until the PVS mixture presents as a substantially uniform color without streaks.

14. The method of claim 10, further comprising the step of rolling the PVS mixture into an elongated tubular shape.

15. The method of claim 10, further comprising the step of holding the impression tray filled with the PVS mixture in place on the one of the upper dental arch or the lower dental arch for at least three minutes before removing the impression tray.

16. The method of claim 10, further comprising the step of drying the PVS mixture after forming the first dental impression.

17. The method of claim 10, further comprising the step of ejecting the light body PVS over the first dental impression within 45 seconds of opening the single-use dispenser.

18. The method of claim 10, further comprising the step of removing the impression tray from the one of the upper dental arch or the lower dental arch about 1 minute to about 4 minutes after reseating the impression tray to form the second dental impression.

19. The method of claim 10, further comprising the step of placing a cheek retractor into a mouth of a user.

20. The method of claim 19, further comprising the step of taking a picture of the mouth of the user after the cheek retractor is placed in the mouth of the user.

21. The method of claim 10, further comprising the step of thermally activating the PVS mixture.

22. The kit of claim 1, wherein a working time of the PVS mixture is controlled in part by thermal activation at a body temperature.
